(19) United States
(12) Patent Application Publication
(10) Pub. No.: US 2026/0077120 A1
(43) Pub. Date: Mar. 19, 2026

(54) INFUSION PUMP ERROR DETECTION AND ALERT COMMUNICATION SYSTEMS AND METHODS

(71) Applicants: BAXTER INTERNATIONAL INC., Deerfield, IL (US); BAXTER HEALTHCARE SA, Glattpark (Opfikon) (CH)

(72) Inventors: Base V. Paul, Hawthorn Woods, IL (US); Raymond Kan, Lake Forest, IL (US); Timothy Hoh, Toronto (CA)

(21) Appl. No.: 19/330,448

(22) Filed: Sep. 16, 2025

Related U.S. Application Data
(60) Provisional application No. 63/695,736, filed on Sep. 17, 2024.

(51) Int. Cl. A61M 5/145; A61M 5/142

(52) U.S. Cl. CPC A61M 5/145; A61M 2005/14208; A61M 2205/18; A61M 2205/3553; A61M 2205/502; A61M 2205/52

(57) ABSTRACT

A system includes at least one infusion pump, an EMR server, an analysis engine, and a communication server. The analysis engine is configured to receive programming parameters and patient information from at least one of the infusion pump(s) and the EMR server. The analysis engine further configured to use at least one machine learning model configured to detect errors and abnormalities of programming parameters to generate an alert based, at least in part, on the programming parameters and the patient information. The communication server is communicatively coupled to a database and configured to both send and receive information from the infusion pump(s) and the analysis engine. The information received from the analysis engine includes the alert, which is saved in the database and transmitted to the infusion pump(s).

### PRIORITY CLAIM

This application claims priority to and the benefit as a non-provisional application of U.S. Provisional Ser. No. 63/695,736 , filed Sep. 17, 2024, the entire contents of which are hereby incorporated by reference and relied upon.

### BACKGROUND

Infusion pumps facilitate administration of intravenous therapy to patients both in and outside of clinical settings. One type of infusion pump is a Patient-Controlled Analgesia (PCA) pump which is commonly used for administering pain medication intravenously. PCA pumps are programmed to deliver an infusion at a continuous constant rate or can include Patient Control Analgesia (PCA) boluses, boluses like loading dose, clinician bolus, Patient Control Epidural Analgesia (PCEA) boluses, PIEB (Programmable Intermittent Epidural Bolus). To administer an infusion, the user programs the PCA pump. PCA pumps include multiple parameters to be programmed by the user, and each parameter has certain acceptable ranges (lower and upper limits). The parameters are interrelated, which makes programming more challenging and leads to programming errors that can delay therapy administration and lead to patient discomfort due to under dosing and overdosing.

Most infusion pumps in the market today provide a drug library for reporting and detecting certain infusion programming errors. However, using a drug library for detecting and/or reporting infusion programming errors has limitations such as including broadly set limits set which accommodate unusual use cases, producing excessive warnings which result in users ignoring alerts, infrequent updates, lack of customizable updates for patient type, patient condition or therapy sequence, and computational and/or data limitations at the pump. A need accordingly exists for an infusion pump system that provides for flexible and accurate detection and alerting of infusion programming errors.

### SUMMARY

The present disclosure sets forth methods, systems, and apparatuses for information flow between medical devices (e.g., infusion pumps), analysis engines and communication servers for detecting errors and providing alerts. A remote monitoring system is disclosed herein that is easier to update and has more flexible updatability than manually updating hard or soft limits in an infusion pump drug library. The remote monitoring system disclosed herein may be implemented on a computer system with higher computing capabilities than a typical infusion pump, thus providing for more sophisticated monitoring features, faster monitoring response times, and better monitoring results. Further, the remote monitoring system of the present disclosure implements data integrations with other medical or hospital information and data systems which allow for more customized, personalized and sophisticated monitoring and error detection techniques than a static drug library provides.

In light of the disclosure set forth herein, and without limiting the disclosure in any way, in a first aspect of the present disclosure, which may be combined with any other aspect, or portion thereof, a system includes at least one infusion pump, an EMR server, an analysis engine, and a communication server. The analysis engine is configured to receive programming parameters and patient information from at least one of the infusion pump(s) and the EMR server. The analysis engine further configured to use at least one machine learning model configured to detect errors and abnormalities of programming parameters to generate an alert based, at least in part, on the programming parameters and the patient information. The communication server is communicatively coupled to a database and configured to both send and receive information from the infusion pump(s) and the analysis engine. The information received from the analysis engine includes the alert, which is saved in the database and transmitted to the at least one infusion pump.

In another aspect of the present disclosure, which may be combined with any other aspect, or portion thereof, the communication server is configured to send and receive data according to an EXTCOM protocol.

In another aspect of the present disclosure, which may be combined with any other aspect, or portion thereof, the at least one infusion pump includes a first infusion pump and a second infusion pump.

In another aspect of the present disclosure, which may be combined with any other aspect, or portion thereof, the first infusion pump is a syringe pump and the second infusion pump is a large volume pump.

In another aspect of the present disclosure, which may be combined with any other aspect, or portion thereof, the first infusion pump is configured to send information according to a first messaging protocol version and the second infusion pump is configured to send information according to a second messaging protocol version.

In another aspect of the present disclosure, which may be combined with any other aspect, or portion thereof, the system includes a communication driver configured to translate the first messaging protocol version to the second messaging protocol version.

In another aspect of the present disclosure, which may be combined with any other aspect, or portion thereof, the system includes a display console configured to display the alert to a user via a user-controllable web user interface.

In another aspect of the present disclosure, which may be combined with any other aspect, or portion thereof, the web user interface includes a user-selectable alert rating.

In another aspect of the present disclosure, which may be combined with any other aspect, or portion thereof, the analysis engine is integrated into the at least one infusion pump.

In another aspect of the present disclosure, which may be combined with any other aspect, or portion thereof, the analysis engine is hosted in a cloud computing environment.

In another aspect of the present disclosure, which may be combined with any other aspect, or portion thereof, an infusion pump includes a pump user interface that is configured to display the alert to a user.

In another aspect of the present disclosure, which may be combined with any other aspect, or portion thereof, the pump user interface includes a user-selectable rating.

In light of the disclosure set forth herein, and without limiting the disclosure in any way, in a second aspect of the present disclosure, which may be combined with any other aspect, or portion thereof, a system includes an EMR server, an infusion pump having an analysis engine, and a communication server. The communication server is communicatively coupled to a database and is configured to both send information to and receive information from the infusion pump. The analysis engine is configured to (i) receive programming parameters and patient information from the EMR server and/or (ii) access locally available information on the infusion pump. The analysis engine is further configured to use at least one machine learning model configured to detect errors and abnormalities of programming parameters to generate an alert based at least in part on the programming parameters and the patient information or the locally available information on the infusion pump.

In another aspect of the present disclosure, which may be combined with any other aspect, or portion thereof, the alert is an identified error and/or an indication of an optimal therapy input.

In another aspect of the present disclosure, which may be combined with any other aspect, or portion thereof, the identified error is a prescription error.

In another aspect of the present disclosure, which may be combined with any other aspect, or portion thereof, the system includes a display console configured to display the alert to a user via a user-controllable web user interface.

In another aspect of the present disclosure, which may be combined with any other aspect, or portion thereof, the web user interface includes a user-selectable alert rating.

In another aspect of the present disclosure, which may be combined with any other aspect, or portion thereof, the infusion pump includes a pump user interface that is configured to display the alert to a user.

In another aspect of the present disclosure, which may be combined with any other aspect, or portion thereof, the pump user interface includes a user-selectable alert rating.

In another aspect of the present disclosure, which may be combined with any other aspect, or portion thereof, the infusion pump is selected from the group consisting of a PCA pump, a syringe pump, a large volume pump, a linear peristaltic pump, an ambulatory pump, and a multi-channel pump.

In another aspect of the present disclosure, which may be combined with any other aspect, or portion thereof, any of the features, functionality and alternatives described in connection with any one or more of FIGS. 1A to 5F may be combined with any of the features, functionality and alternatives described in connection with any other of FIGS. 1A to 5F.

Additional features and advantages are described in, and will be apparent from, the following Detailed Description and the Figures. The features and advantages described herein are not all-inclusive and, in particular, many additional features and advantages will be apparent to one of ordinary skill in the art in view of the figures and description. Also, any particular embodiment does not have to have all of the advantages listed herein and it is expressly contemplated to claim individual advantageous embodiments separately. Moreover, it should be noted that the language used in the specification has been selected principally for readability and instructional purposes, and not to limit the scope of the inventive subject matter.

### BRIEF DESCRIPTION OF THE FIGURES

FIG. 1A is a block diagram of information flows in a medical environment, according to an example embodiment of the present disclosure.

FIG. 1B is a block diagram of information flows in a medical environment, according to an example embodiment of the present disclosure.

FIG. 2A is a diagram of a medical environment configured for the example method(s), apparatus, and system(s) described herein, according to an example embodiment of the present disclosure.

FIG. 2B is a bock diagram of a medical environment configured for the example method(s), apparatus, and system(s) described herein, according to an example embodiment of the present disclosure.

FIG. 3 shows an example workflow demonstrating an example alert, according to an example embodiment of the present disclosure.

FIG. 4 is a block diagram of an analysis engine, according to an example embodiment of the present disclosure.

FIGS. 5A-5F show example displays of a user interface for selecting, filtering and displaying continuous quality improvement (“CQI”) reports, according to example embodiments of the present disclosure.

### DETAILED DESCRIPTION

The present disclosure relates in general to methods, systems, and apparatuses for information flow between medical devices (e.g., infusion pumps), analysis engines associated with the medical devices, and various servers, databases, gateways and terminals within and providing information to the medical environment. The medical environment may include or otherwise be associated with a communication server and/or gateway, for example the Baxter® IQ Enterprise® gateway, an electronic medical record (“EMR”) server and/or database, a pharmacy server, other third-party servers, and other medical systems. The medical environment may also include a continuous quality improvement (“CQI”) system, which may include one or more servers and databases communicatively coupled to other components and systems within the medical environment. Additionally, the other medical systems may include a hospital information system (“HIS”), which may include one or more servers and databases communicatively coupled to other components and systems within the medical environment. The other medical systems may also include an Admission, Discharge, Transfer (“ADT”) system configured to record movements of patient information within and out of the hospital and transmit the patient information to other systems on the hospital's network.

A patient's health documentation is stored in an EMR. An EMR may have various formats, and while there is no universal format, EMRs typically contains a patient's physical characteristics, measured physiological parameters, diagnostic test results, a summary of medical procedures performed, and clinician notes. Many EMRs also include data transmitted from a medical device, such as an infusion pump, that is providing a therapy to a patient. The data from a medical device can include infusion therapy data or renal failure therapy data.

Data and information in an EMR may be auto-populated or updated from a medical device. For example, an EMR server may store data (directly to a patient's EMR) that is received from a medical device (e.g., an infusion or dialysis device). However, this auto-populated data and/or automatic storage may require that a clinician provide an association between the medical device, patient, and medication order (or medical record) prior to providing treatment. For example, to make an association, a clinician has to use an interface of a device to open a medication order from a patient's EMR. The clinician may then enter a patient identifier and/or medical device identifier into the medication order to create an association. In other examples, a clinician may manually enter data or information, such as infusion or dialysis data (e.g., infusion parameters), into an EMR. Infusion parameters may include an infused medication and/or drug, dose information, infusion rate, volume to be infused (“VTBI”), infusion time, infusion schedule, etc.

The infusion pump error detection and alert communication systems, methods and techniques disclosed herein advantageously addresses limitations with existing therapy monitoring techniques. For example, most infusion pumps in the market today provide a drug library for reporting and detecting certain infusion programming errors. However, using a drug library for detecting and/or reporting infusion programming errors has some limitations.

First, the limits in a drug library may often be set to allow every possible (e.g., even extremely rare) use cases, while also warning for programming selections and entries that may be outside of the safest value of ranges, which may result in users ignoring all the warnings (as they are triggered too often) and thus ignore critical warnings for selections and/or entries that well exceed normal use ranges. Second, drug libraries may be updated infrequently and the updates or adjustments may not be based on any actual usage patterns, thereby potentially making the updates less effective or less customized. Third, drug libraries may have limits and parameters that are set based on a specific care area and/or medication. Therefore, the limits and parameters set within the drug library may be the same regardless of an actual patient type (e.g., pediatric vs. adult), a patient condition (e.g., having or not having elevated blood pressure, having or not having an elevated blood glucose level), or a therapy sequence (e.g., specific series of drugs being infused). Therefore, the checks and limits in the drug library that are applied for detecting may be applied without any additional context about the patient type, patient condition, or therapy sequence and thus may not provide personalized, optimized or accurate error reporting for a patient according to their specific infusion programming sequence. Fourth, programming reporting and/or detection on small medical devices, such as an infusion pump, may be limited by the lack of computational facilities and capabilities of the infusion pump, the lack of information available at the device level (e.g., patient condition), or a combination thereof.

The solutions described herein advantageously provide a remote monitoring system that is typically easier to update and has more flexible updatability than manually updating hard or soft limits in an infusion pump drug library. For example, updates may be handled centrally and pushed out remotely. Furthermore, more sophisticated monitoring features, faster monitoring response times, and better monitoring results may be achieved when implementing the monitoring system remotely on computer systems with higher computing capabilities than an infusion pump. For example, the higher computing power of the remote system may provide for more sophisticated monitoring techniques that utilize AI and/or ML on an infusion pump regardless of the local computational capabilities of the infusion pump. The remote system also advantageously allows for integrations with other medical or hospital information and data systems (e.g., EMR and ADT) to get more dynamic information about a patient, a patient therapy, an infusion therapy or treatment, or an infusion program. These data integrations allow for more customized, personalized and sophisticated monitoring and error detection techniques than a static drug library provides.

Each of the advantages or additional capabilities described above may assist with reducing potentially harmful programming errors by alerting a user when a program input (e.g., programmed dose) is outside of acceptable soft or hard limits. The alerts may be provided in a meaningful and consistent manner to protect patient safety and prevent alert fatigue.

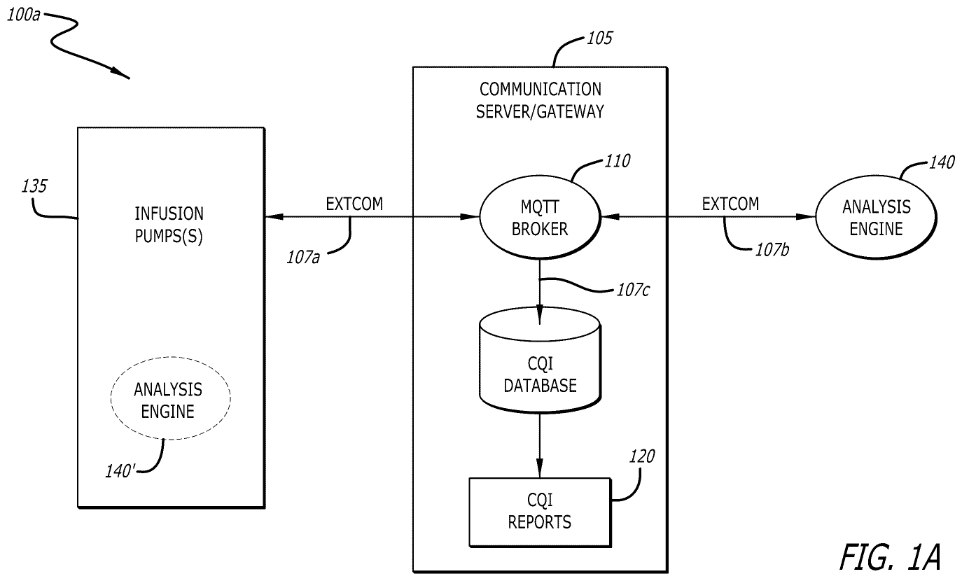

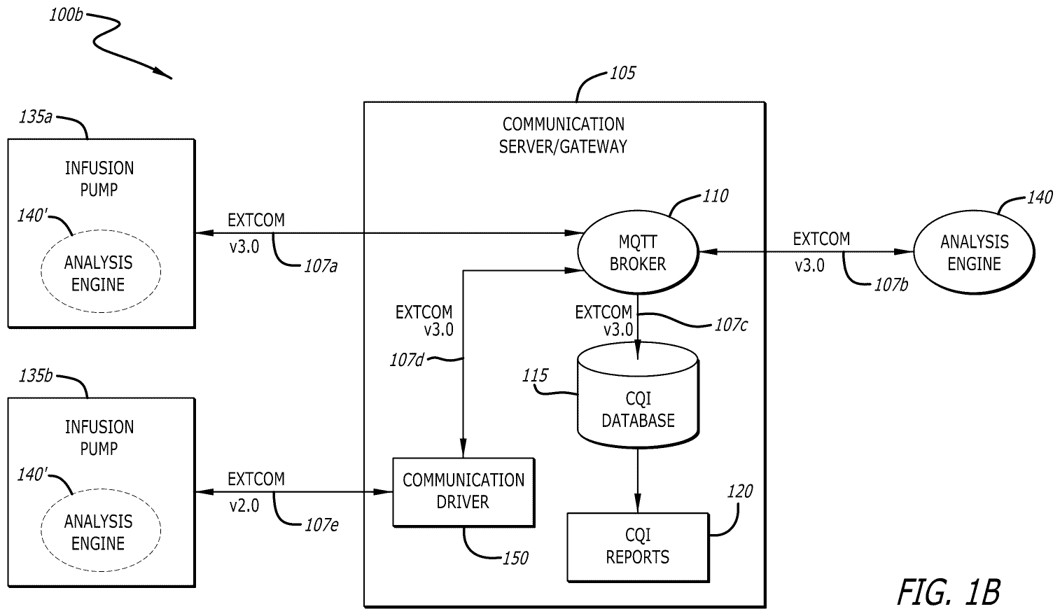

Referring now to the drawings and in particular to FIGS. 1A and 1B, which illustrate high level block diagrams of information flows in a medical environment, which form part of an infusion pump communication system(s) 100A and 100B. In the example illustrated in FIG. 1A, the communication system 100A includes one or more infusion pump(s) 135, a communication server and/or gateway 105, for example the Baxter® IQ Enterprise® gateway, and an analysis engine 140. Specifically, as illustrated in FIG. 1A, the system 100A includes at least one infusion pump 135 and an analysis engine 140 that communicate with the communication server and/or gateway 105. The communication server and/or gateway 105 may use a standards-based messaging protocol, such as the Message Queuing Telemetry Transport (“MQTT”) messaging protocol. For example, communication server and/or gateway 105 may use the MQTT protocol for checking connection status and routing information via a MQTT broker 110 to a continuous quality improvement (“CQI”) database 115, which may be configured to generate, output or provide CQI reports 120.

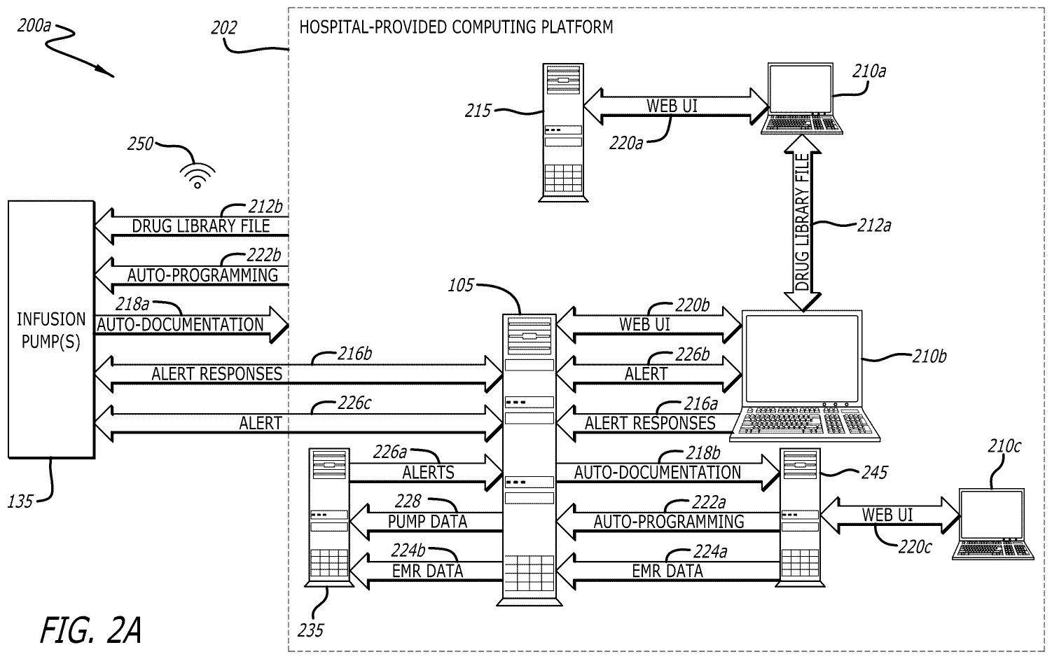

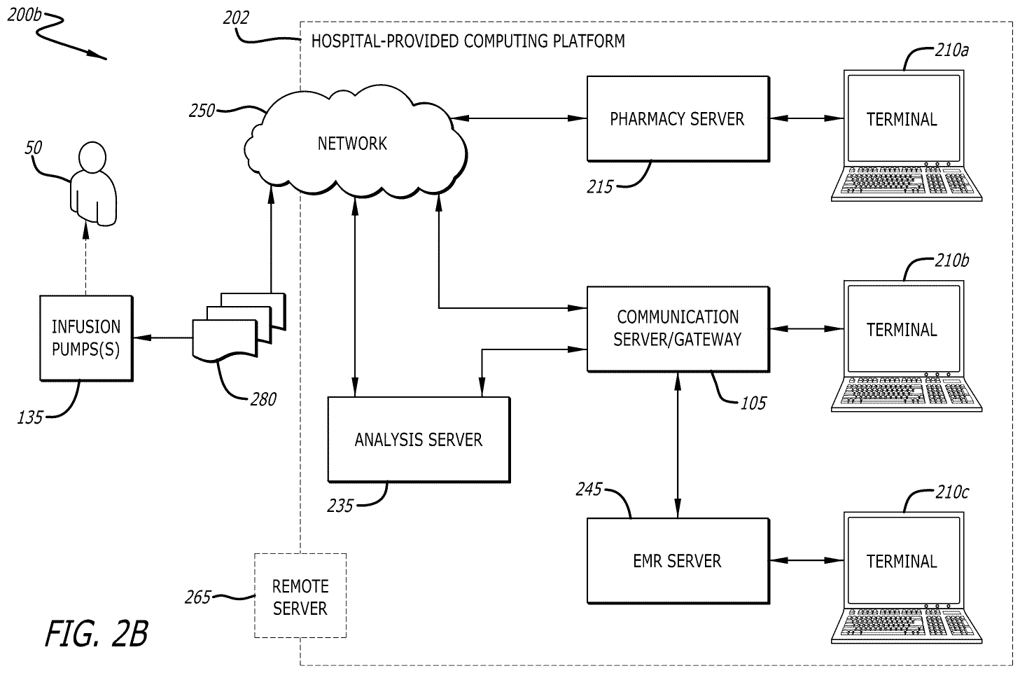

Communication server/gateway 105 may use routing tables, which contain path routing information, and forwarding tables, which contain port information when communicating and sending information to other network components. For example, the routing tables and forwarding tables may be used while routing the traffic or forwarding data to the appropriate destination port. Examples of the type of data routed and forwarded are described in more detail below in FIG. 2A.

MQTT is a standards-based messaging protocol, or set of rules, used for machine-to-machine communication. Smart sensors, wearables, and other Internet of Things (IoT) devices typically have to transmit and receive data over a resource-constrained network with limited bandwidth. Devices may use MQTT for data transmission, as it is easy to implement and can communicate IoT data efficiently. MQTT supports messaging between devices to the cloud and the cloud to the device. The MQTT is an OASIS standard messaging protocol that is designed as an extremely lightweight publish/subscribe messaging transport that is ideal for connecting remote devices with a small code footprint and minimal network bandwidth.

The analysis engine 140 may be configured to plug-in securely to the communication server/gateway's 105 messaging infrastructure. Additionally, the communication server/gateway 105 is configured to send and receive information, illustrated as information flows 107a, 107b and 107c, in an EXTCOM format. In other examples, the communication server/gateway 105 may be configured to send and receive information in an INTCOM format.

In another example, analysis engine 140 may be hosted in a cloud-computing environment. A cloud computing environment provides on demand availability of computer system resources like processing and storage as well as computing services, like software, analysis, and analytics. In the examples illustrated herein, server(s) may be cloud servers and database(s) may be cloud databases that are deployed, delivered, and accessed in cloud. For example, cloud server(s) may be virtual (e.g., non-physical) servers running in a cloud computing environment. Cloud server(s) operate like physical servers and perform similar functions, such as storing data and running applications. Cloud database(s) may organize and store structured, unstructured, and semi-structured data like a traditional on-premises database.

In another example, all or part of the analysis engine 140 may be part of or run locally on an infusion pump(s) 135. For example, as illustrated in FIGS. 1A and 1B, all or part of an optional analysis engine 140′ may be part of or may run locally on the infusion pump 135. The infusion pump(s) 135 may communicate and cooperate with other medical devices and/or devices from the cloud computing environment, such as remote servers and databases.

In the example illustrated in FIG. 1B, the communication system 100B includes a communication driver 150, in addition to each of the components described above with respect to FIG. 1A. The one or more infusion pump(s) 135 of FIG. 1A are shown as two different infusion pumps 135a and 135b in FIG. 1B. Additionally, the added components result in additional information flows, illustrated as information flow 107d and 107e.

The infusion pump(s) 135 or medical pumps may facilitate administration of intravenous therapy to patients both in and outside of a clinical setting. One type of medical pumps inside the clinical setting is a Patient-Controlled Analgesia (PCA) pump. PCA pumps typically deliver pain medication and may allow patients to request medication delivery (e.g., a bolus) from the PCA pump or may be pre-programmed to deliver intermittent boluses by programming the PCA pump to deliver a specific bolus volume with a specific time interval between two boluses. Other types of medical pumps include infusion pumps, such as a syringe pump, a linear peristaltic pump, a large volume pump (“LVP”), an ambulatory pump, a multi-channel pump, etc. The infusion pump(s) 135, may include a commercially available PCA pump, LVP, syringe pump or any other infusion pump, such as such as the Baxter Novum IQ or Baxter Spectrum IQ.

As noted above, sending and receiving information in FIGS. 1A and 1B, for example the information flows 107, may use the EXTCOM protocol. EXTOM uses MQTT over Transport Layer Security (“TLS”) as the underlying transport protocol to carry application-specific data. The application-specific data objects follow a Json syntax. The EXTCOM protocol may have different versions and the implemented version may depend on the model of infusion pump. For example, a first type of infusion pump 135a, such as a Baxter Novum IQ pump, may use a first version (e.g., v3.0) of the protocol while a second type of infusion pump 135b, such as a Baxter Spectrum IQ pump, uses a second version (e.g., v2.0) of the protocol. In one example, Baxter's Novum IQ pumps may make use of version 3.0 of the EXTCOM protocol while Spectrum IQ pumps make use of version 2.0 of the EXTCOM protocol, and the communication driver 150 may be configured to update and/or normalize any data in the information flow such that all information received, processed and routed by the MQTT Broker 110 is of the same protocol version (e.g., v3.0). The communication driver 150 may serve as a translation layer that translates older versions of EXTCOM protocol to the latest version or a predetermined version (e.g., v3.0).

Specifically, information sent from infusion pump 135b along information flow 107e may be converted by communication driver 150 from a second version (e.g., v2.0) to a first version (e.g., v3.0) before sending the information to the MQTT broker 110 via information flow 107d. Similarly, information sent from the MQTT broker 110 to infusion pump 135b may be routed through the communication driver 150 to covert the information from EXTCOM v3.0 to EXTCOM v2.0.

It should be appreciated that the systems, methods and techniques described herein may be communication technology agnostic and as such, other variants of remote communication technologies may be used. Additionally, any of the wired connections described herein may be wireless and any of the wireless connections may instead be wired. Furthermore, different or other communication protocols may be used (e.g., TCP, UDP, TLS, MQTT, HTTP, etc.) to send and receive information.

The analysis engine 140 may serve as a near real-time remote monitoring and alert system. For example, the analysis engine 140 may be configured to identify errors or other abnormal scenarios and then provide alerts based on the errors or abnormalities identified. In an example, the analysis engine 140 may identify errors and/or abnormalities through machine learning (“ML”) models and ML training, which is described in more detail below. In other examples, the analysis engine 140 may be manually configured to identify errors and/or abnormalities, which may be provided as soft and hard coded limits, analysis files, or other manual data entry and configuration techniques. In yet other examples, the analysis engine 140 may identify additional, updated or optimal inputs for treatment and therapy programming (e.g., an optimal therapy input).

Specifically, it should be appreciated that even though many of the examples described herein relate to identifying errors or other abnormal scenarios and then providing alerts based on the errors or abnormalities identified, the systems, methods and techniques described herein may also be applied to identifying new or more optimal inputs for therapy programming and providing an alert or an update based on the updated therapy inputs. As noted in more details below, in some examples the one or more ML models, or a portion of an ML model may be implemented locally at the infusion pump to perform at least a portion of the monitoring and/or identifying errors.

Conversely, the systems, methods and techniques disclosed herein advantageously allow an infusion pump to send messages and/or programming events real-time (or near real-time) to a remote server such that the messages may be received remotely by an analysis engine along with other dynamic information (e.g., patient condition) from other sources (e.g., EMR server). It should be appreciated that “real-time” communication of an event (e.g. a programming event) may only be a requirement for “real-time” alert display on an infusion pump screen, and that the remote capabilities of the analysis engine 140 may function with delayed data (e.g., a pump may be operating while not connected to a network) and may provide alerts or other review information offline. Additionally, the analysis engine 140 The remote analysis engine advantageously provides configurability to update error reporting conditions and what conditions are considered abnormal. Additionally, errors or alerts may be reported to the infusion pump when abnormal values or sequences are detected, thereby preventing therapy errors or providing a more optimal treatment.

The analysis engine 140 may be configured to receive messages that an infusion pump 135a or 135b, generally referred to herein as infusion pump(s) 135, sends to the communication server/gateway 105. In an example, analysis engine 140 is configured to identify potential prescription errors with infusion pump(s) 135 and communication server/gateway 105. Error identification may be achieved by a machine learning model employed by the analysis engine 140. The machine learning model may be configured to detect errors and abnormalities of programming parameters. For example, the machine learning model may analyze the parameters programmed on the infusion pump(s) based on data including abnormal and/or normal programming values and/or sequences in order to identify errors or abnormalities in the programming parameters. The analysis engine 140 is adapted for use within an infusion programming workflow, rather than responding to medication orders received from an in-hospital EMR. For example, analysis engine 140 is configured to analyze the received messages without requiring any additional software development for the infusion pumps or communication server/gateway 105. Depending on the analysis, an alert may be generated by the analysis engine 140 and saved in a CQI database 115. Additionally, the generated alerts may be displayed to a user through a CQI report 120. In other examples, the generated alerts may be transmitted to the infusion pump(s) and/or displayed at the infusion pump(s) 135, e.g., via a pump user interface (“UI”). The user may then respond to the alerts (e.g., accept or override warnings provided by the alerts) through the pump UI and the user responses may be sent back to the analysis engine 140.

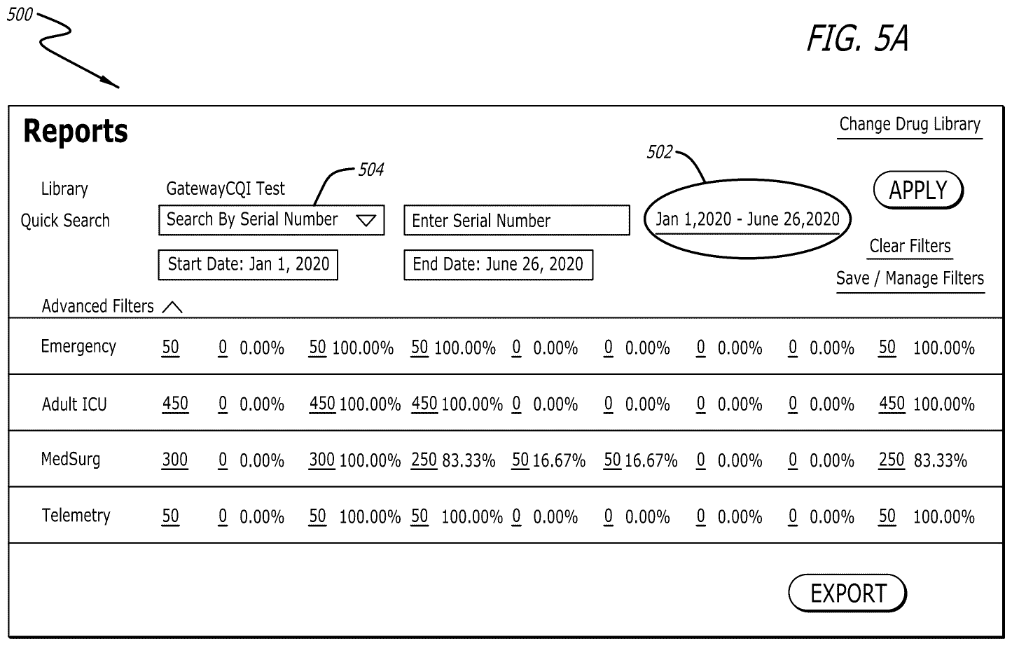

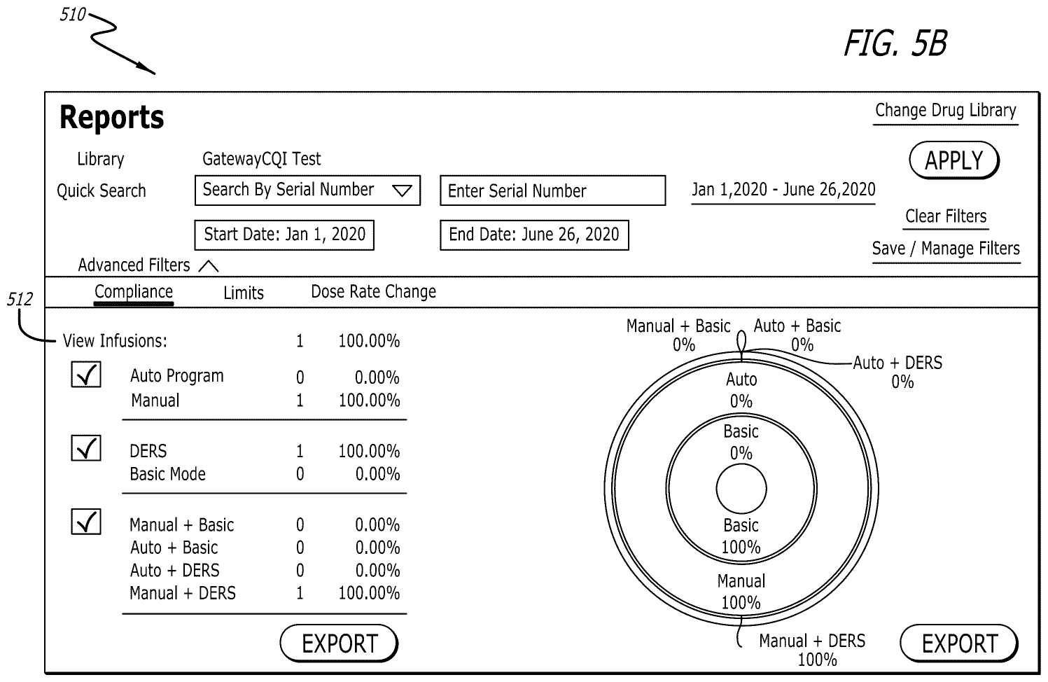

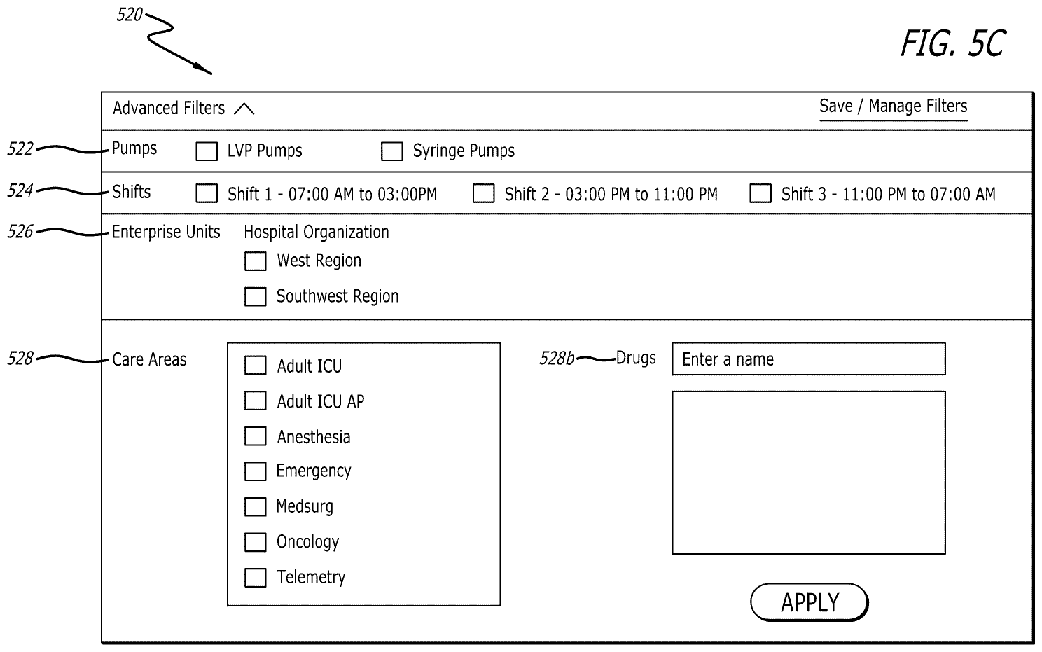

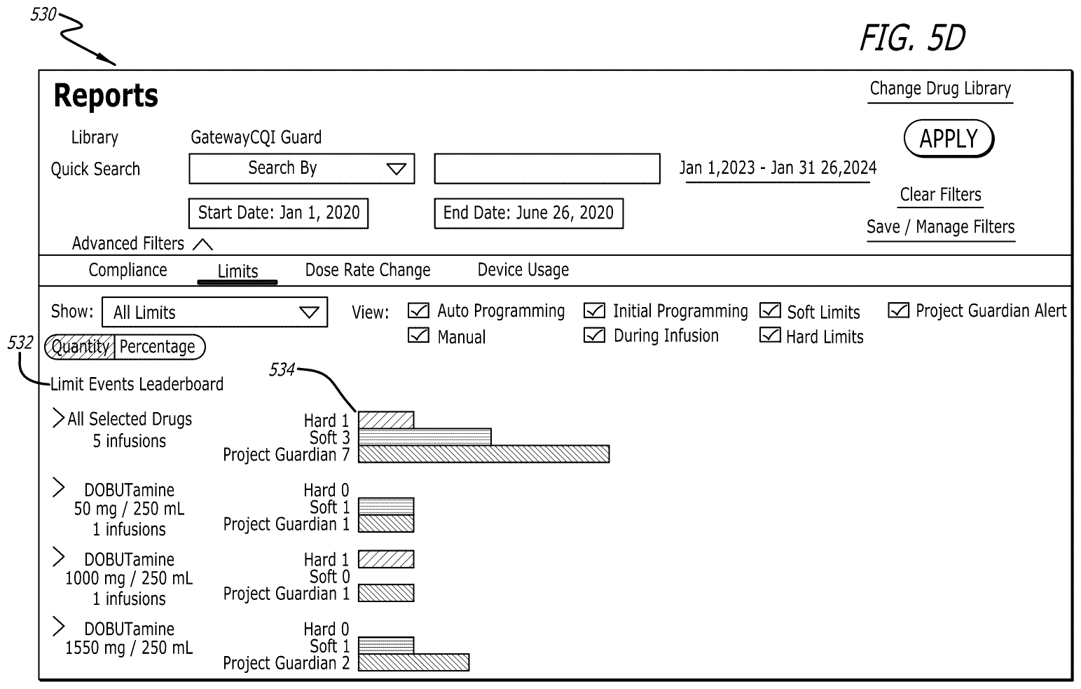

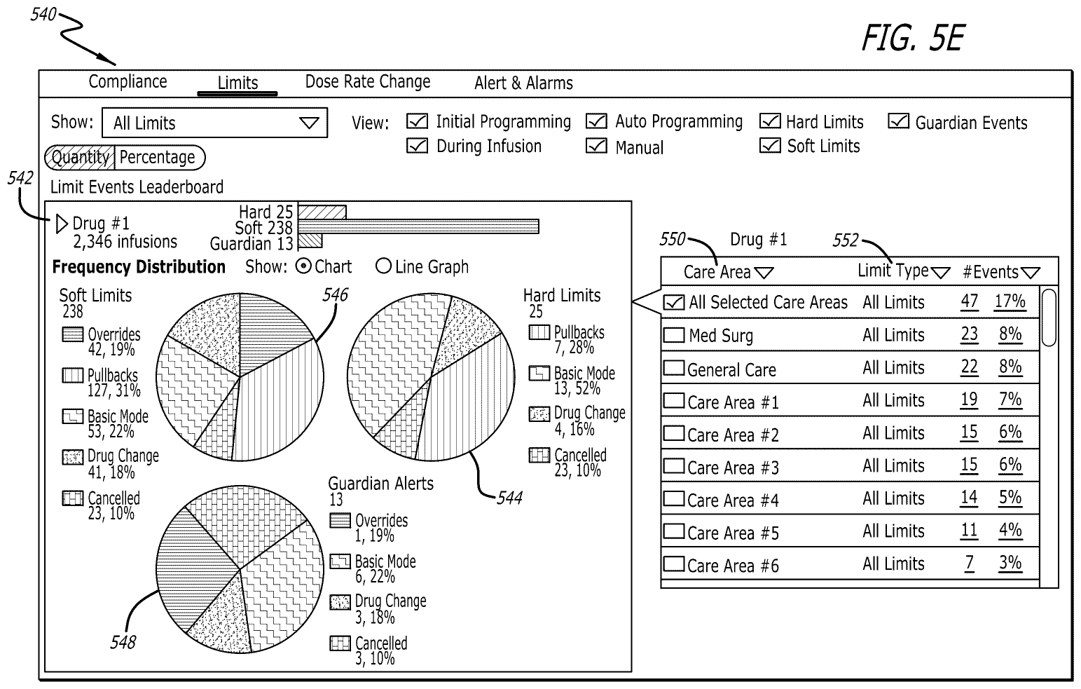

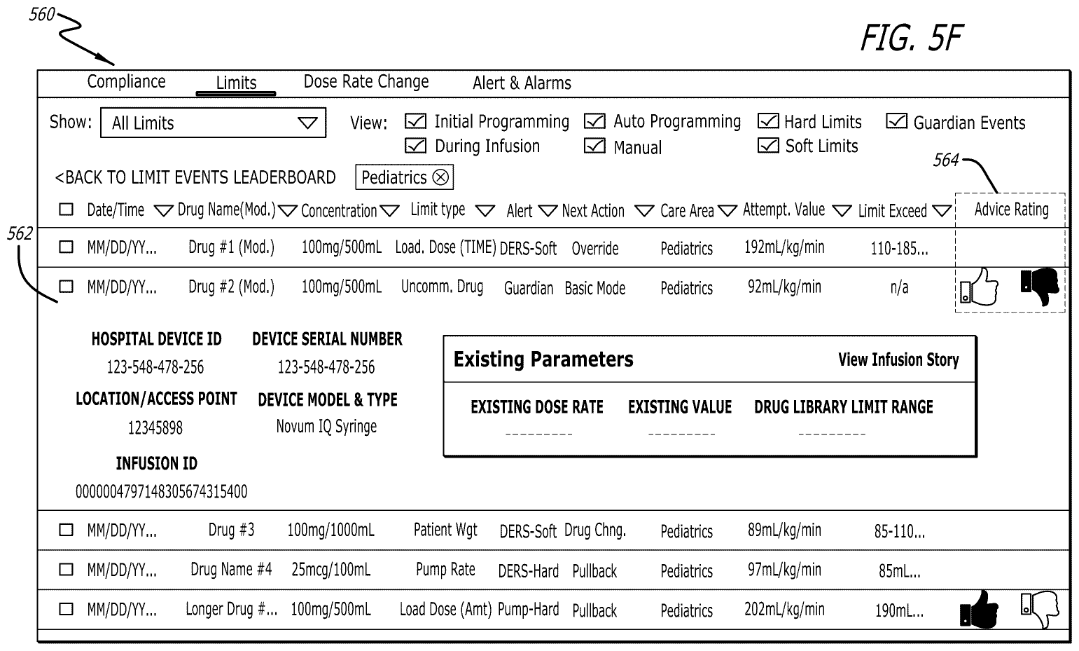

In an example, a CQI report 120 may be generated and displayed with integrated user controls such that the report is reviewable by a user and configured for receiving user feedback, which is described in more detail below in relation to FIG. 5F. For example, a CQI report 120 may include controls for users to review and provide feedback to the analysis engine 140, for example to indicate whether an alert being reviewed is appropriate or inappropriate. The user feedback may then be used to update and/or refine the analysis engine 140, such as a ML model employed by the analysis engine 140.

Analysis engine 140 may be deployed as a standalone hardware or software component that interacts with various infusion pump(s) 135 and communication server and/or gateway 105, and more specifically, the CQI reports 120. Analysis engine 140 may also be a hardware or software component integrated with the infusion pump(s) 135. It should be appreciated that both the infusion pump(s) 135 and communication server and/or gateway 105 are capable of operation without the analysis engine 140 present, installed and/or enabled. For example, the analysis engine 140 may be disabled periodically to perform updates without affecting the operational functionality of the infusion pump(s) 135 or communication server/gateway 105.

Referring now to FIGS. 2A and 2B, which illustrates a diagram of a medical environment configured for the example method(s), apparatus, and system(s) described herein, the systems 200A, 200B include a medical environment or hospital-provided computing platform 202 includes communication server/gateway 105, pharmacy server(s) 215, analysis server(s) 235, EMR server(s) 245, various terminals 210a-c, and associated databases (not pictured).

FIG. 2A shows specific data and information flows between components, while FIG. 2B shows a higher-level block diagram of the communication channels, paths or couplings. Also, FIG. 2B shows a patient 50 receiving an intravenous therapy from the infusion pump(s) 135. Infusion pump(s) 135 may include any pump capable of delivering an intravenous therapy to the patient 50 via one or more line sets. Examples include a syringe pump, a linear peristaltic pump, a large volume pump (“LVP”), an ambulatory pump, multi-channel pump, etc. A syringe pump uses a motor connected to a drive arm to actuate a plunger within a syringe. A linear peristaltic pump uses a rotor to compress part of a tube while rotating. As mentioned above, example infusion pumps include Baxter Novum IQ or Baxter Spectrum IQ pumps.

Infusion pump(s) 135 is communicatively coupled to the various components of the hospital-provided computing platform 202, and more specifically communication server/gateway 105 via a network 250. Data 280 may be received by or sent from infusion pump(s) 135. The example communication server/gateway 105 is configured to send data to and receive data from infusion pump 135. The sent data may include drug library files 212b, prescription data, auto-programming information 222b, and software updates. Data received by communication server/gateway 105 may include medical device data and other auto-documentation data 218a and route the data to an EMR server 245. The infusion pump(s) 135 may include a memory storing one or more drug libraries (e.g., from the drug library files 212b) that include drug library limits, which may be particular program parameter limits based on care area, dose change, rate of change, drug type, concentration, patient age, patient weight, etc. The limits are configured to ensure that a received prescription or entered infusion therapy is within acceptable ranges and/or limits decided by a medical facility, doctor, or clinician. These limits and whether they were exceeded, and actions that followed exceeding the limits, may form at least part of a limits report, which is described in more detail below with respect to FIG. 5D. In some embodiments, the communication server/gateway 105 via a network 250 is configured to convert the data from, for example, EXTCOM message(s) to HL7 message(s).

In the examples illustrated in FIGS. 2A and 2B, each server may be communicatively coupled to a database. For example, EMR server 245 may be communicatively coupled to an EMR database (not pictured) for storing EMR records. The EMR database may also be configured to store infusion pump data. The infusion pump data may be pump specific instead of patient specific (e.g., data that is not associated to a particular patient and/or medication order). The EMR server 245 is also communicatively coupled to a pharmacy server 215, which is configured to create and/or transmit medication orders corresponding to, for example, prepared medications. A medication order may include an electronic record or entry, which identifies a patient (e.g., a patient identifier) and infusion parameters for administration. Some example infusion parameters included in a medication order may include an infused medication and/or drug, dose information, infusion rate, volume to be infused (“VTBI”), infusion time, infusion schedule, etc.

The example network 250 may include any wired or wireless connection (e.g., an Ethernet network, LAN, WLAN, etc.). Furthermore, any of the databases described herein may be stored in any volatile or non-volatile memory device including RAM, ROM, flash memory, magnetic or optical disks, optical memory, or other storage media. The database(s) may be structured as a relational database or a graph database. If the database includes a graph database, patients, medication orders, medication device data, and identifiers may be provided by separate nodes.

The hospital-provided computing platform 202, and more specifically communication server/gateway 105 may also be configured to transmit auto-programming information 222b, drug library file 212b and other operating or prescription parameters to the infusion pump(s) 135. The auto-programming information 222b may be received at select screens on a pump UI in the pump workflow. For example, the communication server/gateway 105 may send auto-programming information 222b and/or a drug library file 212b, such as an electronic prescription to the infusion pump(s) 135 at a predetermined time and/or when the infusion pump(s) 135 is available to accept the prescription. In other instances, the infusion pump(s) 102 may be configured to periodically poll the communication server/gateway 105 to determine if any drug library file(s), electronic prescription(s), or perhaps even a software update(s) are awaiting to be downloaded to the pump.

In an example, the pharmacy server 215 and associated database (not pictured) may communicate and share data with a workstation, display, console or terminal 210a through a web user interface (“UI”) 220a. As used herein “terminal” may refer to a workstation, display, consol or computer configured for displaying and providing functionality (e.g., communication functionality) with another terminal, server, etc. through a web UI. The terminal 210a may send drug library information, such as a drug library file 212a to another consol or terminal 210b that interacts with communication server/gateway 105 through web UI 220b. As mentioned above with reference to FIGS. 1A and 1B, an alert (e.g., alert 226 of FIG. 2A) may be generated by the analysis engine 140 and saved in a CQI database 115. Additionally, the generated alerts may be displayed to a user through a CQI report 120, which may be displayed terminal 210b. In another example, generated alerts may be transmitted to and/or displayed directly on an infusion pump(s) 135, for example an infusion pump 135 may have a display screen configured for displaying the alert which has been received from the analysis engine 140.

Pharmacy server 215 may be configured to provide web-based drug library access, titration error prevention, etc. For example, the pharmacy server 215 may provide access to drug library files (e.g., drug library file 212a), which may be provided to a terminal 210a via web UI 220a. The drug library file 212a may be provided to infusion pump(s) 135 (as drug library file 212b) via the hospital-provided computing platform 202, and more specifically through communication server/gateway 105. For example, drug library file 212a may be provided to communication server/gateway 105 via another terminal 210b, which as noted above may be a workstation, display console or the like, such as a computer.

EMR server(s) 245 may communicate, send or otherwise provide EMR data 224a and auto-programming data 22a to communication server/gateway 105, which similar to the drug library file 212a may be provided to infusion pump(s) 135 as auto-programming data 222b. The EMR server(s) 245 may receive auto-documentation data 218b from communication server/gateway 105, which was originally provided by infusion pump(s) 135 (illustrated as auto-documentation data 218a). It should be appreciated that auto-documentation data 218a and 218b may be the same or may be adjusted and formatted differently as it is sent and received by various components of the system 200A. Similarly, it should be appreciated that EMR data 224a and 224b as well as the drug library file 212a and 212b may be the same or may be adjusted and formatted differently based on the component sending the information.

In an example, web UI(s) 220a, 200b and 200c may provide step-by-step workflows and prompts for a user at the associated terminal(s) 210a, 210b and 210c.

The analysis server 235 and/or analysis engine 140 may receive pump data 228 and EMR data 224b, which may be analyzed to determine an error, such a prescription error. Once identified, an alert 226a may be generated for the identified prescription error and sent by analysis server 235 to communication server/gateway 105, which may be conveyed to terminal 210b as alert 226b. The alert (e.g., alert 226c) may also be conveyed to the infusion pump(s) 135. In the illustrated example, the alert 226c may be sent to the infusion pump(s) 135 via the communication server/gateway 105. The error, such as a potential prescription error, may be identified based on a ML model employed by the analysis engine 140, which is communicated to the analysis server 235. The analysis server 235 may be communicatively coupled to analysis engine 140. In another example, analysis server 235 may be part of analysis engine 140 or vice versa. The analysis server 235 and/or analysis engine 140 may receive messages, data (e.g., pump data 228 and EMR data 224b) or other information that the infusion pump(s) 135 send to communication server/gateway 105 (e.g., pump data 228) along with EMR data 224a sent by EMR server 245, analyze the information, and generate an alert if an error is identified. The alert is eventually provided and displayed to the user, e.g., through a CQI report 120, on terminal 210b. The user may review and provide feedback regarding the alert 226b and provide an alert response 216a indicating whether the alert was appropriate.

As illustrated in FIG. 2B, a medical environment or hospital-provided computing platform 202 includes communication server/gateway 105, pharmacy server(s) 215, analysis server(s) 235, EMR server(s) 245, and various terminals 210a-c. The servers and/or gateways may be associated with databases.

In the examples described above, infusion pump(s) 135 send information to communication server/gateway 105 and receives information, such as alert information, from the communication server/gateway 105. As noted above, alert information or an alert (e.g., alert 226c) may be sent to the infusion pump(s) 135 from the communication server/gateway 105. However, in other examples, the infusion pump(s) may receive alerts directly from analysis engine 140. The alert 226c may be displayed on a user interface (“UI”) of the infusion pump(s) 135 thereby allowing a user to accept or override the warning provided by the alert 226c. Additionally, infusion pump(s) may be configured to send acknowledgements to at least one of the communication server/gateway 105 and the analysis engine 140 regarding the received alert. Based on the user's choice and/or actions (e.g., overriding the warning), the alert response(s) 216b may be sent to the communication server/gateway 105 and/or conveyed to the analysis engine 140 where the user feedback (e.g., alert responses(s) 216b) may then be used to update and/or refine the analysis engine 140, such as a ML model employed by the analysis engine 140.

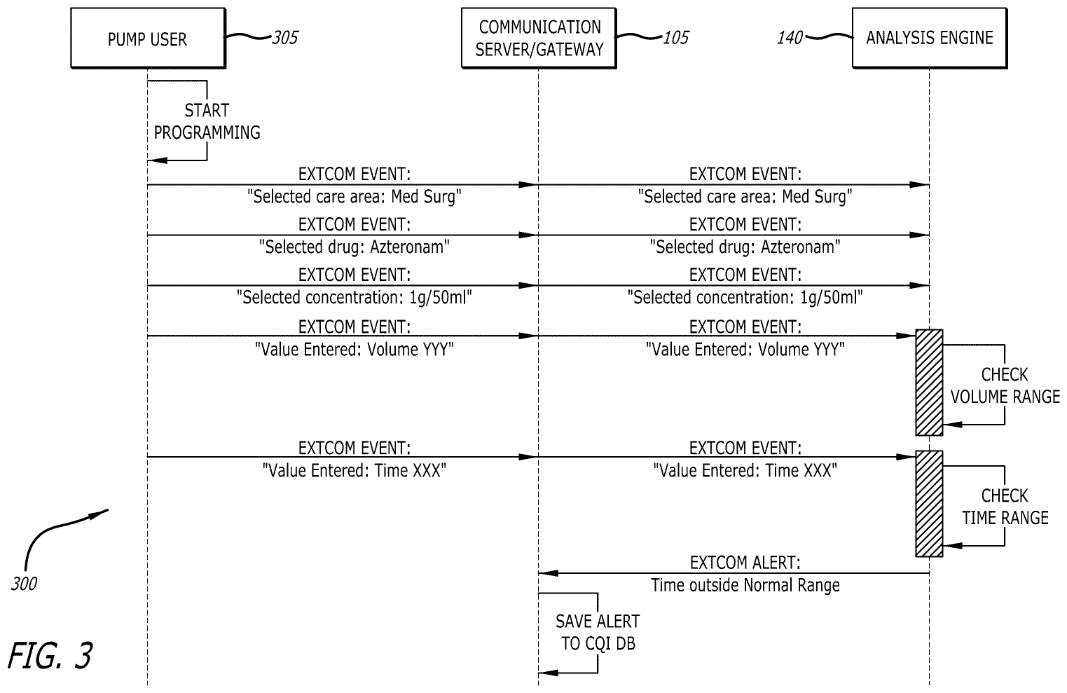

Referring now to FIG. 3, which shows an example workflow 300 demonstrating an example alert, includes information flows between a pump user 305, communication server/gateway 105 and analysis engine 140. As illustrated in FIG. 3, the workflow starts with pump user 305 initiating or starting programming a therapy on an infusion pump 135. The programming may include selecting a care area (e.g., “Med Surg”), which is communicated to the communication server/gateway 105 and later conveyed to analysis engine 140. The therapy may also include a selected drug (e.g., “Aztreonam”) a selected concentration (e.g., “1 g/50 mL”) and a volume to be infused (e.g., “YYY”). The selected drug, selected concentration, and the volume information are sent to the communication server/gateway 105 and then forwarded or otherwise provided by the communication server/gateway 105 to analysis engine 140.

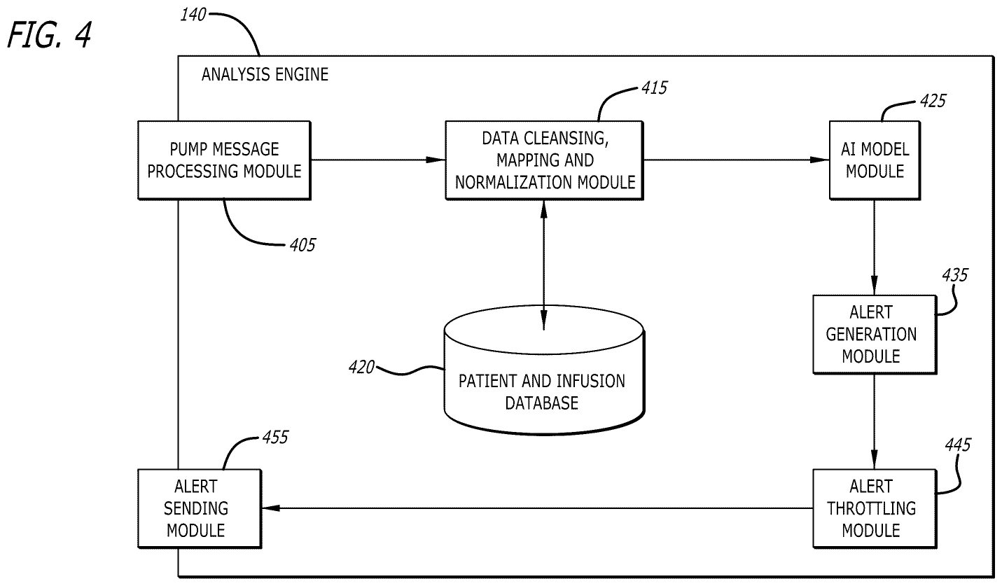

Then, analysis engine 140 analyzes the received information to check the volume range. In the illustrated example, the volume range is acceptable and no errors are detected. The pump user 305 enters a treatment time (e.g., “time XXX”), which is communicated to communication server/gateway 105. Then, the communication server/gateway forwards the information to analysis engine 140. Analysis engine 140 analyzes the received information to check the time range. In the illustrated example, the analysis engine detects an error with the selected (e.g., programmed) time range and sends an alert indicating “time outside normal range” to the communication server/gateway 105. The communication server/gateway saves the alert to the CQI database 115. Once saved in the CQI database, the alert may be provided in a CQI report 120 and submitted for review and approval. In another example, the alert may be provided directly at the infusion pump(s) 135 (e.g., on a pump UI) where the user may review and respond to the alert (e.g., accept, override, make therapy adjustment, etc.). The user's alert response may then be communicated to the analysis engine 140 where it may be used to update and/or refine the analysis engine 140. Using the alert responses to update and/or refine the analysis engine 140 may be performed periodically (e.g., not automatically). In the illustrated example in FIG. 4, the information is communicated in an EXTCOM format using the EXTCOM protocol.

Error detection may be based on manually set limits or parameters, similar to programming or setting limits with a Drug Library. The analysis engine 140 may be manually configured to identify errors and/or abnormalities, which may be provided as soft and hard coded limits, analysis files, or other manual data entry and configuration techniques. For example, the analysis engine may detect an error with the selected (e.g., programmed) time range based on acceptable time range limits or parameters that were manually set or pre-programmed. Then, when the selected (e.g., programmed) time range is outside of the manually set or pre-programed range, the analysis engine may send an alert indicating “time outside normal range”to the communication server/gateway 105.

In other examples, error detection may occur at the device level (e.g., at the infusion pump 135). In some examples, error detection may occur at the device level (e.g., at the infusion pump 135) with the assistance or in conjunction with a remote server, such as pharmacy server(s) 215, analysis server(s) 235, EMR server(s) 245 of FIGS. 2A and 2B. In another example, the infusion pump(s) 135 may provide error detection in conjunction with and in communication with another remote server 265 of FIG. 2B, which may or may not be part of the medical environment or hospital-provided computing platform 202. In some examples, the remote server may provide AI and/or ML information and/or models to the infusion pump(s) 135.

Communication with one or more of the communication server/gateway 105, pharmacy server 215, analysis server 235, EMR server 245, or other remote server(s) 265 allows for enhanced error detection and monitoring techniques and strategies. For example, as noted above, error detection may be based solely on manually set soft and hard coded limits on the infusion pump 135. By providing connectivity to network 250 and to other devices, components and servers, monitoring and error detection may be expanded to multiple infusions, such as multiple infusion pumps, multiple infusion therapies, etc. By providing access to additional information and data, such as patient history data (e.g., patient information and data, lab results, other pharmacy and treatment information), the error detection and monitoring techniques may be based on relation limits and analysis from the various data inputs.

Regarding the various enhanced versions of remote monitoring and error detection described above, the capabilities may depend on the level of cross-device and network communication and information availability. For example, at a basic level, a simple version of monitoring and error detection may involve real-time (or near real-time) detection of an error or abnormality and integrating that error alert into a respective report. A more enhanced version may include displaying the error alert in real-time on the infusion pump screen. Furthermore, another version may be further sophisticated in detection to take into consideration all of the infusions delivered to a patient (either concurrently or in sequence) instead of just the instant infusion. To further enhance monitoring and error detection, additional information may be considered such as patient's condition.

FIG. 4 illustrates a block diagram of an example analysis engine 140. In the illustrated example, analysis engine 140 includes a pump message processing module 405, a data cleansing, mapping and normalization module 415, a patient and infusion database 420, an AI model module 425, an alert generation module 435, an alert throttling module 445, and an alert sending module 455.

It should be appreciated that the analysis engine 140 and error detection techniques may be implemented on more than one computer or computation resource. Additionally, multiple hardware and/or software applications may be used to accomplish the monitoring and error detection techniques disclosed herein.

The pump message processing module 405 may be configured to receive messages, data and/or information from an infusion pump 135 and/or communication server/gateway 105. The received messages, data and/or information are then sent to the data cleansing, mapping and normalization module 415 to be processed. The processing may involve cleaning the data, mapping the data to an appropriate table (which may involve including data from the patient and infusion database 420), and normalizing or reformatting the data such that the data can be analyzed by the AI model module 425.

In an example, patient physiological and/or demographic information my be received from the EMR Server (e.g., EMR server 245 of FIGS. 2A, 2B) or the patient and infusion database 420, such as gender, weight, Care Area, etc. The patient physiological information along with the data and/or information from the infusion pump (e.g., after being cleaned, mapped and normalized at block 415) may be processed and analyzed by AI model module 425 to determine any relationships between patient physiological information and/or demographic information and infusion programs the breach safety limits. For example, a certain patient demographic or patient physiological parameter may be associated with outlier events, e.g. instances where the attempted therapy included unusual infusion program parameters. These program parameters may have been due to user error, or a unique set of circumstances based on a specific set of physiological and/or demographics that had a tendency to create a scenario where a therapy would be outside of one or more limits.

In other instances, the AI model module 425 may be able to identify trends or find patterns associated with therapy outliers based on pump types, hospital shifts, Care Areas, or specific drugs. For example, a specific pump type may be unsuitable for therapy with a specific drug in some edge cases (e.g., for patients of a certain weight). In other instances, several outliers may be identified for a specific hospital shift, which may indicate additional training is required. In other scenarios. In other instances, a certain Care Area or specific drug may be correlated with several unusual infusion program parameters, which may indicate that the auto-programming for that Care Area or drug may need to be reviewed and adjusted.

Each instance of an attempt to program an unusual infusion parameter, an anomaly, or a limit being hit or exceeded may be identified by the analysis engine 140, and more specifically AI model module 425. The identification and relationships built with the data obtained by analysis engine 140 may be provided in a searchable, filterable and reviewable alert or report. For example, the information obtained at block 425 may assist with monitoring compliance with established clinical procedures, identify unusual infusions that may merit further investigation, or indicate when the programming practices for a particular drug might need to be reviewed.

Limits reports, one example of which is illustrated in FIG. 5D, provides a user the ability to analyze and understand the frequency of infusion programs that breach drug library limits or drug and Care Area specific safety limits, which may be defined by customer pharmacists in drug libraries. For example, a limits report may detail instances where pump users attempted to program highly unusual infusion program parameters that significantly exceed typical ranges and thus may include medication errors. The report may help identify potentially problematic drugs and limits, investigate pump user behavior, and improve limits in clinical practice.

Once analyzed and processed by the AI model module 425, which may include various AI models, algorithms, and processes to review and analyze information received from the data cleansing, mapping and normalization module 415.

The AI models may be standardized statistical models that are refined based on historical data from the care facility via machine learning and artificial intelligence in order to highlight combinations of pump infusion programming parameters and care area context information that are statistical outliers. AI model module 422 outputs may be used to generate reports that identify unusual infusion parameters, which may assist users in making informed improvements to drug library settings. The reports may also assist users in understanding the occurrence of unusual infusion program selections that, if appropriate, may be address via enhanced clinical training.

The output from the AI model module 425 is then passed to the alert generation module 435, which is configured to generate an alert if the output indicates an error exists. Then, the alert throttling module 445 may classify and/or prioritize the alert based on the severity of the error. Once the alert is classified and/or prioritized, the alert information is passed to the alert sending module 455, which is configured to communicate the alert to the communication server/gateway 105 where it can be presented to a user for review at a terminal (e.g., terminal 210b).

In many of the examples discussed above, the AI and/or ML models are described as being implemented remote from the infusion pump(s) 135. However, it should be appreciated that the AI and/or ML models may also be implemented locally on a medical device, such as an infusion pump 135. Referring briefly back to FIGS. 1A and 1B, infusion pump 135 is illustrated with an optional analysis engine 140′, which may include all or portions of any of the analysis engine 140 examples described herein. For example, AI and/or ML models may run locally on the infusion pump 135 to provide monitoring and error detection capabilities. An AI and/or ML model (e.g., an initial model, an updated model, or a further fined model) may be pushed to the infusion pump 135 periodically, which may provide updates to the infusion pump monitoring and error detection techniques, such as adjusting and/or adding error hard and soft limits, adding new error scenarios to monitor for, adjusting and/or adding error detection and analysis techniques (e.g., comparisons, determinations and/or calculations). Specifically, the AI and/or ML model(s) pushed to the infusion pump 135 where analysis occurs locally on the infusion pump based on locally available information on the pump (e.g., any current pump operating information such as infusion rate, flow rate, and any other patient or treatment information that is programmed into the pump).

In the event the AI and/or ML models are implemented locally on the infusion device, the model learning (e.g., model adjustment, updates, refinements and/or enhancements) may still be performed remotely, as described in the various examples above. For example, model implementation may be local at the infusion pump 135, but model generation and learning may be remote.

### Report Generation and Filtering

FIGS. 5A-5F show example displays of a user interface for selecting, filtering and displaying CQI reports. The reports may be sorted, filtered or refined for review and analysis based on several factors including, but not limited to, infusion parameters, care areas, and limit types.

Infusion parameters may include infused medication and/or drug, dose information, infusion rate, volume to be infused (“VTBI”), infusion time, etc. When setting up a therapy, infusion parameters may be populated from a patient's order in the EMR.

Care areas may refer to the care area or location in a care facility, in which a medical therapy is administered. An example set of care areas may include anesthesia, critical care, intensive care unit (“ICU”), medical surgery (“MedSurg”), labor and delivery, oncology, etc. Other medical facilities may have different care areas based on their care specialty or care offerings. For example, care areas may include additional areas other than those listed above, such as general healthcare, nutritional care, kidney care, respiratory care, pharmacy care, primary care, surgical care, neonatal care, etc. Care areas may be further classified by patient's age or weight (e.g., “Adult Critical Care” or “Infant Critical Care”).

Limits may include soft limits and hard limits. Hard and soft limits may exist for concentration, patient weight or body surface area (“BSA”), dose, loading dose, amount or time, pump rate, bolus. Limits and associated limits reports are described in more detail with respect to FIG. 5D.

FIG. 5A shows a first display 500 of a user interface for selecting generated CQI reports 120 from a navigation bar of infusion pump applications. Once selected, the user may select a drug library to view reports in the search window or in the report display window. Reports may be generated or retrieved for specific date ranges 502. For example, the first display 500 illustrates reports from Jan. 1, 2020 to Jun. 26, 2020. In other examples, a search may be conducted for a specific infusion pump to retrieve reports related to that infusion pump's usage. The user may perform a infusion pump search 504 to search for reports related to a specific infusion pump. The infusion pump search 504 may include searching by device serial number as illustrated in the first display 500.

FIG. 5B shows a second display 510 of a user interface illustrating a report 512 showing usage data for a specific pump. Reports may be sorted and filtered. In one example, the reports may be sorted to identify a specific infusion pump to visualize usage data for that pump instead of searching through an entire fleet of pumps, as seen in the second display 510. In the report 512 of the second display 510, the infusion information (e.g., “view infusions”) is displayed, which shows the infusion pump was used for one (1) manual infusion.

FIG. 5C shows a third display 520 for filtering reports. As illustrated in the third display 520, reports can be filtered by pump type 522 (e.g., LVP Pumps, Syringe Pumps, etc.), hospital shifts 524 (e.g., first shift from 7:00 AM to 3:00 PM, second shift from 3:00 PM to 11:00 PM, and third shift from 11:00 PM to 7:00 AM), enterprise units or hospital regions 526, care areas 528 (e.g., adult ICU, Adult ICU AP, Anesthesia, Emergency, MedSurg, Oncology, Telemetry, etc.), and administered medications/drugs 528b. The medications and/or drugs 528b may be searchable by name. For example, the reports may allow users may review visual depictions of each infusion run, including starts, stops, infusion rates, volumes infused, and events that occur including any limits that are hit or overridden.

Limits that are hit or overridden may be part of a limits report. Limits may depend on or be established for patient weight, patient BSA, rate, infusion time, VTBI, drug amount, dose, concentration, concentration volume, etc. Some example limits include a drug concentration limit, which are applicable for variable concentration drugs. Typically, a hitting or reaching a soft limit may provide an alert or notification to the user that the limit was hit, and the user can ignore the limit (e.g., press “yes” to proceed) or may adjust the selected or entered amount (e.g., press “no” and re-enter amount). As discussed in more detail below, information about the limits (e.g., upper, lower hard or soft) hit or exceeded along with the subsequent user response or action may be included in a limits report.

For example, a drug concentration limit may have a soft limit if the drug amount and volume entered exceed a lower or upper concentration soft limit configure for the drug (e.g., user notified “Above upper concentration limit of 4 mg/mL, Accept 4.5 mg/mL?”). The user may choose to proceed with the entry that is above the soft limit or make an adjustment to the therapy. If a drug concentration hard limit is hit or exceeded, reprogramming the treatment or therapy may be required (e.g., user notified “Above UPPER Hard concentration limit of 5 mg/mL, re-check and re-enter values”).

Another example limit is a patient weight/BSA limit, which may have established hard and soft limits for the patient's weight or BSA for a particular Care Area. Example notifications for exceeding a patient weight soft limit may be “Above upper limit of 150 kg, accept 200 kg?” and an example alert or notification for exceeding a patient weight hard limit may be “Above UPPER Hard Limit, enter a value of 300 kg or lower.” Similarly, dose or rate may have both hard and soft limits for lower and upper bounds of dose and/or rate.

Loading dose limits may indicate if a loading dose amount or time entered exceeds lower or upper limits (either soft limits or hard limits) configured for a drug. Amount and/or time limits may be configured for an amount of drug administered or time of administration. The Amount/time limits may indicate if the amount or time entered exceeds lower or upper limits (either soft limits or hard limits) configured for a drug. An example message for a drug (e.g., Vancomycin) being exceeded may be “Soft Limit: below lower limit of 250 mg, accept 200 mg?” with an option of “Yes” or “No” selectable by a user. The same drug (e.g., Vancomycin) for Critical Care may have an upper hard limit of 2000 mg. Time change limits may result when the time is changed with a rate and VTBI already programmed, and a time change alert or notification may display the corresponding change in dose rate or infusion rate (e.g., “the entered time will decrease the rate from 50 mL/hr to 41.7 mL/hr, accept change?”).

Other limits, such as pump rate limits may have a rate field that will display (“LOW”) in red text when the dose entered results in a calculated rate that is lower than the pump rate hard limit, similarly the rate field may display (“HIGH”) in red text when the dose entered results in a calculated rate that is higher than the pump rate hard limit. Bolus limits may be based on a bolus dose amount or time entered and whether the entered dose amount or time entered exceeds a lower or upper limit configured for a drug. Single step dose or rate change limits may be based on a particular facility's drug library. For example, each facility's drug library may be programmed with limits for the percentage of a dose or rate change made in one programming step for each specific drug. In some examples, the dose or rate change for Anesthesia or other Care Areas may be set to 500 percent, such that any dose or rate change that exceeds 500 percent may result in a dose change alert or notification.

FIG. 5D shows a fourth display 530 of a user interface illustrating a limits report 532. An example limits report 532 provides a user the ability to analyze and understand the frequency of infusion programs that breach drug library limits or drug and Care Area specific safety limits, which may be defined by customer pharmacists in drug libraries. For example, a limits report 532 may detail instances where pump users attempted to program highly unusual infusion program parameters that significantly exceed typical ranges and thus may include medication errors. The report may help identify potentially problematic drugs and limits, investigate pump user behavior, and improve limits in clinical practice.

In the example limits report 532 illustrated in FIG. 5D, the quantity of occurrences 534 that a dose limit was attempted during an infusion is displayed as well as the quantity of occurrences a user attempted to program highly unusual infusion parameters. The limits report can be further filtered to show a breakdown of infusion by Care Area.

Furthermore, as shown in the fifth display 540 of FIG. 5E, portions of a report 542 may be expanded to show additional details, such as what a user chose to do after attempting a limit violation for hard limits 544, soft limits 546, and other alerts 548 (e.g., override limit warnings and proceed with infusion beyond Soft limits, pull back and proceed with the infusion within limits, pull back and cancel the infusion and then ran a basic mode infusion, pull back and cancel the infusion to either perform an infusion of a different drug or no other infusion at all). Pullbacks may indicate potentially avoided mistakes while other subsequent actions may indicate user error or non-compliance with established clinical procedures.

The user's subsequent actions may help determine if the selection or entry was inadvertent or by mistake (e.g., user chose to make a change to the value) or if the selection was intentional (e.g., user chose to override the limit warning). Intentional items may indicate that the programming practices for a particular drug might need to be reviewed.

Reports may also be filtered to display infusion parameter distribution graphs of the infusions run using a selected drug. The distribution graphs may plot the frequency of different program parameters that were run using a specific drug, which may provide a visual representation of user programming behavior to help identify problem areas. Specifically, the shape of a resulting frequency distribution may help a user quickly identify a problem area, such as a drug library setting or a need to update a specific clinical practice. For example, one may expect a bell-shaped curve that tails down as limits are reached and a curve that is flat or approaches limit with increasing frequency may identify trends that warrant further investigation. The distribution graphs may be filtered to display data from a specific care area 550. The display may also be filtered by limit type 552, whether the limit event took place before or during the infusion, and whether or not it was auto-programed as illustrated in FIG. 5E. These additional filters may provide additional perspective and representations of the data to identify any trends and possible corrective actions.

FIG. 5F shows an interface 560 where the user is able to provide feedback or a response (e.g., alert response 216a and 216b of FIG. 2A) to determine accuracy or usefulness of an alert 562. In the illustrated example, the feedback 564 is a “thumbs-up” or a “thumbs-down” but may also be a numerical rating, scale or other selection to indicate how accurate or useful the alert was. This feedback or response may be used to improve the analysis engine 140 and associated machine learning algorithms over time. By providing user input to rank, score or gauge that validity of alerts, the analysis engine 140 may advantageously be improved and fine-tuned to provide more accurate alerts. The feedback may also be used to customize the analysis engine 140 for a specific medical facility (e.g., hospital), specific equipment (e.g., certain model or brand of infusion pump), specific Care Area and/or specific hospital practices.

Providing positive feedback (e.g., thumbs-up), may indicate that the alert provided by analysis engine 140 is useful and the alert appropriately identified an undesired infusion parameter. Providing negative feedback (e.g., thumbs-down) may indicate that the alert is not useful, for example perhaps the unusual infusion parameter is a normal occurrence or at least well within standard practices at this specific facility or Care Area. By providing negative feedback, these false positives or otherwise unnecessary alerts may be avoided in the future.

The reports may also provide an “infusion story”, which gives a graphical representation of the history of an infusion, including infusion start and stop times, infusion rates, volume infused, occurrence of drug limit and events, and occurrence of analysis engine alerts.

It should be understood that various changes and modifications to the presently preferred embodiments described herein will be apparent to those skilled in the art. It is therefore intended that such changes and modifications be covered by the appended claims.

The invention is claimed as follows:

1. A system comprising:
at least one infusion pump;
an EMR server;
an analysis engine configured to receive programming parameters and patient information from at least one of the at least one infusion pump and the EMR server, the analysis engine further configured to use at least one machine learning model configured to detect errors and abnormalities of programming parameters to generate an alert based at least in part on the programming parameters and the patient information; and
a communication server communicatively coupled to a database, the communication server configured to both send and receive information from the at least one infusion pump and the analysis engine, wherein
the information received from the analysis engine includes the alert,
the alert is saved in the database, and
the alert is transmitted to the at least one infusion pump.

2. The system of claim 1, wherein the communication server is configured to send and receive data according to an EXTCOM protocol.

3. The system of claim 1, wherein the at least one infusion pump includes a first infusion pump and a second infusion pump.

4. The system of claim 3, wherein the first infusion pump is a syringe pump and the second infusion pump is a large volume pump.

5. The system of claim 3, wherein the first infusion pump is configured to send information according to a first messaging protocol version and the second infusion pump is configured to send information according to a second messaging protocol version.

6. The system of claim 5, further comprising a communication driver configured to translate the first messaging protocol version to the second messaging protocol version.

7. The system of claim 1, further comprising a display console configured to display the alert to a user via a user-controllable web user interface.

8. The system of claim 7, wherein the web user interface includes a user-selectable alert rating.

9. The system of claim 1, wherein the analysis engine is integrated into the at least one infusion pump.

10. The system of claim 1, wherein the analysis engine is hosted in a cloud computing environment.

11. The system of claim 1, wherein an infusion pump of the at least one infusion pump includes a pump user interface that is configured to display the alert to a user.

12. The system of claim 11, wherein the pump user interface includes a user-selectable alert rating.

13. A system comprising:
an EMR server;
an infusion pump having an analysis engine; and
a communication server communicatively coupled to a database, the communication server configured to both send information to and receive information from the infusion pump, wherein
the analysis engine is configured to at least one of (i) receive programming parameters and patient information from the EMR server and (ii) access locally available information on the infusion pump, and
the analysis engine further configured to use at least one machine learning model configured to detect errors and abnormalities of programming parameters to generate an alert based at least in part on the programming parameters and the patient information or the locally available information.

14. The system of claim 13, wherein the alert is one of (i) an identified error and (ii) an indication of an optimal therapy input.

15. The system of claim 14, wherein the identified error is a prescription error.

16. The system of claim 13, further comprising a display console configured to display the alert to a user via a user-controllable web user interface.

17. The system of claim 16, wherein the web user interface includes a user-selectable alert rating.

18. The system of claim 13, wherein the infusion pump includes a pump user interface that is configured to display the alert to a user.

19. The system of claim 18, wherein the pump user interface includes a user-selectable alert rating.

20. The system of claim 13, wherein the infusion pump is selected from the group consisting of a PCA pump, a syringe pump, a large volume pump, a linear peristaltic pump, an ambulatory pump, and a multi-channel pump.
